(19) United States
(12) Patent Application Publication
(10) Pub. No.: US 2023/0233733 A1
(43) Pub. Date: Jul. 27, 2023

(54) PHOTOCATALYTIC STRUCTURE CAPABLE OF UNIFORMIZING ULTRAVIOLET LIGHT

(71) Applicant: SHENZHEN YITOA INTELLIGENT INDUSTRIAL CO., LTD, Shenzhen City (CN)

(72) Inventors: JUNHUANG ZHUANG, SHENZHEN CITY (CN); ZHIHONG ZHENG, SHENZHEN CITY (CN)

(21) Appl. No.: 17/717,047

(22) Filed: Apr. 9, 2022

(30) Foreign Application Priority Data
Jan. 25, 2022 (CN) 202220207781.1

(51) Int. Cl. A61L 9/20; F21V 7/00

(52) U.S. Cl. CPC A61L 9/205; F21V 7/005; F21V 7/0083; A61L 2209/12; F21Y 2105/16

(57) ABSTRACT

A photocatalytic structure that can uniformize ultraviolet lights is provided. The photocatalytic structure includes a housing, a groove formed at a top of the housing, multiple grille slices being engaged with two sides of an inner wall of the groove, multiple grille passages being formed between every two adjacent grille slices, metal meshes disposed on both top and bottom of the grille slices, and a UVC-LED light board disposed at a side of the grille slices. The photocatalytic structure stacks the grille slices to form the grille passages. The UVC-LED light board makes grille passages to reflect the ultraviolet light. More uniform lights are formed at both upper and lower sides of the grille passages. The UVC-LED light board close to the metal meshes ensures the uniform lights to be emitted onto a gap plane with a smaller refraction angle. Therefore, everywhere on the metal meshes are fully illuminated.

### CROSS-REFERENCE TO RELATED PATENT APPLICATION

This application claims the benefit of priority to Patent Application No. 202220207781.1, filed on Jan. 25, 2022 in People's Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure is related to a technical field of photocatalysis, and more particularly to a photocatalytic structure that is capable of uniformizing ultraviolet ray.

### BACKGROUND OF THE DISCLOSURE

Air sterilization and disinfection indicates a process of disinfection for microorganisms and bacteria in the air. Most of the conventional air sterilization-and-disinfection devices adopt an ultraviolet lamp combined with a photocatalytic mesh to enhance the effect of disinfection and sterilization of the air.

The photocatalyst is a light catalyst that is a general term for semiconductor materials represented by nano-scale Titanium dioxide capable of photocatalysis. The representative photocatalyst is Titanium dioxide that is able to produce strong oxidative substances under light irradiation, and can be used to decompose organic compounds, parts of inorganic compounds, bacteria and viruses, etc. In daily life, the photocatalyst can effectively degrade toxic and harmful gas in the air so that the air can be well sterilized. The toxic and harmful gas is such as a formaldehyde. The air can be efficiently purified. In the meantime, a variety of bacteria can be effectively killed. The toxin released by the bacteria or the fungus can be decomposed and detoxified.

However, for ensuring that the conventional air sterilization-and-disinfection device makes the ultraviolet ray being irradiated onto a photocatalytic mesh fully, the ultraviolet lamp is required to keep a distance from the photocatalyst. However, the ultraviolet ray cannot irradiate the photocatalyst fully even though a larger area where the photocatalyst acts can provide better effect of sterilization. Accordingly, the conventional air sterilization-and-disinfection device cannot make the photocatalyst module acting a maximum effect in a best way.

### SUMMARY OF THE DISCLOSURE

A photocatalytic structure of the present disclosure is provided for solving shortcomings of the conventional technologies. The photocatalytic structure can uniformize ultraviolet rays. In the photocatalytic structure, a plurality of grille passages are formed by stacking multiple grille slices. A UVC-LED light board is disposed on each of the grille slices. Each of the grille passages can reflect the ultraviolet ray. More uniform light emitting surfaces are formed on both upper and lower sides of the grille passage. Because the UVC-LED light board closes to the metal mesh, it is ensured that the light irradiates a gap plane of the metal mesh with a uniform and smaller refraction angle. Therefore, everywhere on the metal meshes are fully illuminated.

For implementing the above technical purpose, the present disclosure provides a photocatalytic structure that can uniformize ultraviolet ray. The photocatalytic structure includes a housing, a groove openly disposed on a top of the housing, multiple grille slices engaged with two side of an inner wall of the groove, a plurality of grille passages disposed between every two adjacent grille slices, two metal meshes disposed on a top and a bottom of the grille slices, and a UVC-LED light board disposed on one side of each of the grille slices.

Further, the metal mesh is in a honeycomb shape.

Still further, one side of the metal mesh is disposed with a photocatalyst.

Further, both two sides of an inner wall of the groove can be disposed with a PCB substrate. The two sides of the UVC-LED light board are engaged with the PCB substrate.

Still further, the photocatalyst is made of Titanium dioxide.

Further, one side of the grille slice is disposed with a reflective layer.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic diagram depicting an overall photocatalytic structure according to one embodiment of the present disclosure;

FIG. 2 is a schematic diagram depicting a housing structure of the photocatalytic structure according to one embodiment of the present disclosure; and

FIG. 3 is a schematic diagram depicting a grille passage of the photocatalytic structure according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The present disclosure is related to a photocatalytic structure that is capable of uniformizing ultraviolet ray and provides beneficial effects as follows.

In one of the beneficial effects of the photocatalytic structure, multiple grille slices are stacked for forming a grille passage, and each of the grille slices is disposed with a UVC-LED light board so that every grille passage can reflect the ultraviolet ray. Accordingly, at least two more uniform light emitting surfaces are formed at both upper and lower sides of the grille passages.

In one further beneficial effect of the photocatalytic structure, the UVC-LED light board is configured to be close to metal meshes and ensure that the lights can uniformly irradiate a gap plane of the metal meshes with a smaller refraction angle. Therefore, everywhere on the metal meshes are fully illuminated.

### EMBODIMENTS

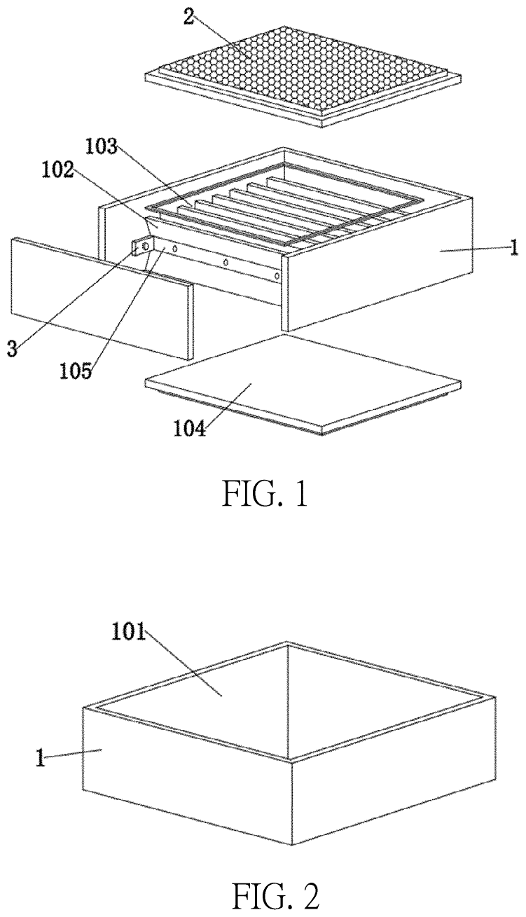

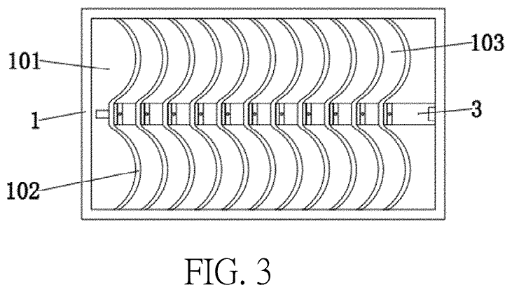

References are made to FIGS. 1-3. The present disclosure provides a photocatalytic structure that is configured to be able to uniformize ultraviolet ray. The photocatalytic structure includes a housing 1 and a groove 101 disposed on a top of the housing 1. The groove 101 is used to mount the multiple grille slices 102 and the multiple grille slices 102 engaged with two sides of an inner wall of the groove 101 form a grille passage 103. The grille passage 103 openly disposed between every two adjacent grille slices 102 acts as a delivery channel for air. The metal meshes 104 disposed at both top and bottom of the grille slices 102 are used to dispose the photocatalyst 2. A UVC-LED light board 105 disposed at one side of the grille slices 102 is configured to emit the ultraviolet ray to sterilize the passing air.

Further, the metal mesh 104 can be in a honeycomb shape. The honeycomb-shaped metal mesh 104 has lower wind resistance and is not affected by wind. The metal mesh 104 is not limited to the appearance of the honeycomb, but can be a shape of square, rectangle or diamond hole. The honeycomb-shaped metal mesh 104 has a larger area than other shapes, and it provides a better effect since the shape attached with the photocatalyst 2 has a larger area.

Further, the photocatalyst 2 can be disposed on one side of the metal meshes 104. When a device having the photocatalytic structure is in operation, the UVC-LED light board 105 provides two bactericidal factors including irradiation sterilization and photocatalyst sterilization. Therefore, the photocatalytic structure enhances the effect of air sterilization and disinfection.

Still further, a PCB substrate 3 can be engaged with two sides of the inner wall of the groove 101. Two sides of each of multiple UVC-LED light boards 105 are engaged with the PCB substrate 3. When the device is in operation, the PCB substrate 3 can supply power to the UVC-LED light boards 105.

Further, the photocatalyst 2 can be made of Titanium dioxide material, and the photocatalyst 2 made of the Titanium dioxide produces strong oxidizing substances if the Titanium dioxide is irradiated. The photocatalyst 2 is therefore able to decompose the organic compounds, parts of the inorganic compounds, bacteria and viruses.

Still further, a reflective layer is disposed at one side of the grille slices 102. When the UVC-LED light board 105 is driven to emit the ultraviolet light, at least one more uniform light emitting surface can be formed on at both upper and lower sides of the grille passages 103. Accordingly, the lights irradiated on the photocatalyst 2 can be more uniform.

In the above embodiments, the description with respect to each of the embodiments has its own emphasis, and parts of some of the embodiments that lack details can refer to the descriptions of other embodiments.

The above embodiments also describe the details of the photocatalytic structure that is capable of uniformizing ultraviolet light of the present disclosure. More particularly, in the photocatalytic structure, multiple grille slices are stacked for forming a grille passage, and each of the grille slices is disposed with a UVC-LED light board so that every grille passage can reflect the ultraviolet light. The lights can uniformly irradiate a gap plane of the metal meshes with a smaller refraction angle and the passing air can be efficiently sterilized and disinfected.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A photocatalytic structure that is able to uniformize ultraviolet lights comprising:
a housing;
a groove formed at a top of the housing;
multiple grille slices engaged with two sides of an inner wall of the groove;
a grille passage formed between every two adjacent grille slices;
metal meshes disposed at a top and a bottom of the grille slices; and
a UVC-LED light board disposed at one side of the grille slices.

2. The photocatalytic structure according to claim 1, wherein the metal mesh is in a honeycomb shape.

3. The photocatalytic structure according to claim 1, wherein one side of the metal mesh is disposed with a photocatalyst.

4. The photocatalytic structure according to claim 3, wherein the photocatalyst is made of Titanium dioxide.

5. The photocatalytic structure according to claim 1, wherein two sides of an inner wall of the groove are disposed with PCB substrates and two sides of the multiple UVC-LED light boards are engaged with the PCB substrate.

6. The photocatalytic structure according to claim 1, wherein one side of the grille slice is disposed with a reflective layer.
